(19) United States
(12) Patent Application Publication
(10) Pub. No.: US 2007/0239254 A1
(43) Pub. Date: Oct. 11, 2007

(54) System for percutaneous delivery and removal of a prosthetic valve

(76) Inventors: Chris Chia, Irvine, CA (US); Rajesh Khanna, Tustin, CA (US); Philippe Marchand, Lake Forest, CA (US); David Taylor, Lake Forest, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/400,063

(22) Filed: Apr. 7, 2006

(51) Int. Cl. A61F 2/06

(52) U.S. Cl. 623/1.11

(57) ABSTRACT

A valve-retrieval device permits a non-deployed valve mounted on a balloon catheter to be retracted back into an introducer sheath for removal from a patient's body. In particular embodiments, the valve-retrieval device is adapted to be placed on a balloon catheter shaft and then advanced over the shaft into the blood vessel via the introducer sheath. The valve-retrieval device has an expandable distal end portion that assumes an expanded shape when advanced out of the introducer sheath. The valve is positioned within or adjacent the distal end portion of the retrieval device, and the retrieval device and the balloon catheter are retracted together back into the introducer sheath. The distal end portion of the retrieval device, rather than the outer surface portion of the valve covered thereby, contacts the distal end and inner surface of the introducer sheath to facilitate retraction of the valve into the introducer sheath.

### FIELD OF THE INVENTION

The present application generally relates to a system for removing implantable devices from body lumens. More particularly, the invention relates to a system for percutaneous delivery and removal of a prosthetic valve, such as a prosthetic heart valve.

### BACKGROUND OF THE INVENTION

Catheters are known in the art and have been commonly used to reach locations inside the body that are not readily accessible by surgery or where access without surgery is desirable. For example, it is known to use a flexible catheter to deliver an implantable device, such a stent or prosthetic valve, through a body lumen, such as the lumens found in the cardiovascular system or gastrointestinal tract.

Prosthetic heart valves have been used for many years to treat cardiac valvular disorders. The native heart valves (i.e., aortic, pulmonary, mitral and tricuspid valves) serve critical functions in assuring the forward flow of an adequate supply of blood through the cardiovascular system. These heart valves can be rendered less effective by calcification as well as by congenital, inflammatory and infectious conditions. Such damage to the valves can result in serious cardiovascular compromise and even death. For many years the definitive treatment for such disorders was the surgical repair or replacement of the native heart valve during open heart surgery. Unfortunately, such surgeries are highly invasive and are therefore prone to many complications. More recently, percutaneous heart valve replacement has emerged as an additional therapy for treating cardiac valvular disorders in a much less invasive manner.

In one minimally invasive method of treating a heart valve, a sheath is introduced into a blood vessel (e.g., a femoral artery or vein) and advanced at least partially toward the implantation site to protect the intimal walls of smaller blood vessels (for example at the iliac bifurcation). A prosthetic valve is mounted on an expandable balloon at the tip of a flexible catheter which is then inserted into the blood vessel via the lumen of the sheath. The catheter is advanced through the blood vessel until the prosthetic valve reaches the implantation site. The balloon at the catheter tip is then inflated to expand the prosthetic valve to its functional size for subsequent implantation at the site of the defective native valve.

During the delivery of the prosthetic valve to the treatment site, the valve is held in a radially compressed condition while contained within the sheath. However, once the prosthetic valve emerges from the sheath, it may expand slightly due to internal forces. As a result, it can be difficult to withdraw the prosthetic valve back into the sheath in the event of an aborted delivery procedure. Consequently, after the valve and the balloon are advanced out of the distal end of the introducer sheath, the valve cannot be easily removed from the body. The size and shape of the valve would induce significant trauma to the surrounding vascular tissue of smaller blood vessels if the valve were to be retracted without using a sheath. Hence, if the valve cannot be successfully delivered to the target implantation site (for example because native valve stenosis prevents proper positioning of the prosthetic valve or the catheter cannot be advanced through the blood vessel to the deployment site), it may be necessary to deploy the prosthetic valve in a benign location or remove the prosthetic valve surgically.

Accordingly, there exists a need for a retrieval device that facilitates the removal of a prosthetic valve or other intravascular implant from a body lumen. It is desirable that such a device allows the implant to be easily withdrawn back into an introducer sheath. It is also desirable that the device be easy to use. It is also desirable that the device be configurable to function in combination with existing delivery systems. The present invention addresses this need.

### SUMMARY OF THE INVENTION

Preferred embodiments of the present invention provide a valve-retrieval device that permits a non-deployed valve mounted on a delivery catheter (e.g., a balloon catheter) to be retracted back into an introducer sheath for removal from the patient's body. The retrieval device is particularly well-suited for retrieving a percutaneously introduced heart valve wherein a relatively smaller ID introducer sheath is used to insert the balloon-mounted valve into the patient's vasculature. The retrieval device also can be used to retrieve other types of prosthetic valves, such as self-expanding valves, or other intravascular devices, such as stents, that cannot be readily retracted back into an introducer sheath once ejected from the sheath into a blood vessel.

In particular embodiments, the valve-retrieval device is adapted to be placed on the shaft of a balloon catheter and then advanced over the shaft into the blood vessel via the introducer sheath. The valve-retrieval device has an expandable distal end portion that assumes an expanded shape when advanced out of the introducer sheath. The distal end portion, when expanded, can be placed in a position covering or surrounding at least a portion of the outer surface of the valve. When the valve is positioned within the distal end portion of the retrieval device, the retrieval device and the balloon catheter are preferably retracted together back into the introducer sheath. The distal end portion of the retrieval device, rather than the outer surface portion of the valve, contacts the distal end and inner surface of the introducer sheath to facilitate retraction of the valve into the introducer sheath.

In one embodiment, the valve-retrieval device has a generally spoon shaped distal end portion that is placed over the valve in the blood vessel. As the retrieval device and the valve are retracted into the introducer sheath, the distal end portion collapses around the outer surface of the valve. In another embodiment, the distal end portion of the removal device comprises a plurality of longitudinally extending valve-engaging members that are radially expandable and contractible toward and away from each other between expanded and collapsed positions. When advanced out of the introducer sheath, the valve-engaging members expand to a diameter greater than the outer surface of the valve to allow at least a portion of the valve to be positioned within the valve-engaging members. As the retrieval device and the valve are retracted into the introducer sheath, the valve-engaging members collapse against the outer portion of the valve.

In another embodiment, the retrieval device is configured for use with a valve-delivery system having a balloon catheter and an outer flexible catheter extending over the balloon catheter. The flexible catheter has an adjustment mechanism operable to adjust the curvature of a distal end portion of the balloon catheter so as to assist in steering or guiding the valve through the patient's vasculature. If it becomes necessary or desirable to remove the valve, the retrieval device is placed on the flexible catheter and the retrieval device is advanced over the flexible catheter through the introducer sheath until the distal end portion extends into the blood vessel. The valve is retracted to engage the inner surface of the retrieval device and then both devices are retracted together back into the introducer sheath.

In an alternative embodiment, the retrieval device is connected to the distal end of the flexible catheter and includes plural, longitudinally extending valve-engaging segments that are radially expandable and contractible toward and away from each other. The valve-engaging segments are resiliently retained in the expanded position. To retrieve and remove a valve, the balloon catheter is retracted to urge the valve against the inner surfaces of the valve-engaging segments, causing them to collapse around at least a portion of the valve outer surface to facilitate retraction of the device into the introducer sheath.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of a valve-retrieval device that is used to retract a non-deployed percutaneous prosthetic valve back into an introducer sheath after being inserted into a blood vessel, according to one embodiment.

FIG. 2 is a fragmentary, top plan view of the valve-retrieval device of FIG. 1.

FIG. 3 is a fragmentary side view of the valve-retrieval device of FIG. 1.

FIG. 4 is a schematic side view of a conventional balloon catheter used to deliver a percutaneous valve.

FIG. 5 is a side view of an introducer sheath assembly, shown partially in section, that can be used to introduce the balloon catheter into a blood vessel.

FIG. 6 is a perspective, exploded view of a loader assembly used to insert and remove the balloon catheter from the introducer sheath assembly.

FIG. 7 is a fragmentary side view of the introducer sheath assembly and the loader assembly, with the loader assembly shown inserted into and secured to the introducer sheath assembly.

FIG. 8 shows the loader cap of the loader assembly placed on the shaft of the balloon catheter prior to inserting the balloon catheter into the loader assembly and the introducer assembly.

FIG. 9 shows the balloon catheter inserted into the loader assembly and the loader cap secured to the loader assembly.

FIG. 10 shows the loader assembly inserted into and secured to the introducer sheath assembly and the valve-retrieval device placed on the balloon catheter and inserted into the introducer sheath assembly.

FIGS. 11A and 11B show the valve-retrieval device being mounted on the shaft of the balloon catheter.

FIGS. 12A-12E illustrate the valve-retrieval device being used to retract a balloon-mounted valve back into an introducer sheath.

FIG. 13 is a perspective view of a valve-retrieval device, according to another embodiment.

FIGS. 14A and 14B are side views of another embodiment of a valve-retrieval device showing valve-engaging members of the device in a radially expanded state (FIG. 14A) and in a collapsed state (FIG. 14B).

FIG. 15 is a side view of another embodiment of a valve-retrieval device.

FIG. 16 is a side view a valve-delivery system shown being used to deliver a prosthetic aortic valve through the aortic arch, according to one embodiment.

FIGS. 17A- 17E illustrate the valve-retrieval device of FIG. 1 being used with the valve-delivery system of FIG. 16 to retract the valve back into an introducer sheath.

FIG. 18 is a perspective view of a valve-retrieval device, according to another embodiment.

FIG. 19 is a cross sectional view of the valve-retrieval device of FIG. 18.

FIGS. 20A-20C illustrate the valve-retrieval device of FIG. 18 being used to retract a valve back into an introducer sheath.

FIG. 21 is a side view of a balloon of a balloon catheter shown partially inflated for facilitating removal of a valve from a patient's vasculature.

### DETAILED DESCRIPTION OF THE PREFERRED EMBODIMENTS

As used herein, the singular forms “a” “an,” and “the” refer to one or more than one, unless the context clearly dictates otherwise.

As used herein, the term “includes” means “comprises.” For example, a device that includes or comprises A and B contains A and B but may optionally contain C or other components other than A and B. A device that includes or comprises A or B may contain A or B or A and B, and optionally one or more other components such as C.

During the percutaneous delivery of a prosthetic valve, such as a prosthetic heart valve, the valve typically is mounted on an expandable distal end portion of a delivery catheter and inserted into a blood vessel via an introducer sheath. For example, the valve can be mounted on an expandable balloon of a balloon catheter. The balloon catheter, with the valve mounted on the balloon, is advanced through the blood vessel toward the deployment site. When the valve is positioned at the deployment site, the balloon is expanded to deploy the valve.

In some cases, the valve cannot be successfully delivered to the deployment site. For example, the balloon catheter may be unable to reach the deployment site through the blood vessel or stenosis of the native valve may prevent proper positioning of the valve. The present disclosure concerns a retrieval device (also referred to herein as a removal device) that permits the non-deployed valve and the balloon to be retracted back into the introducer sheath for removal from the patient's body. The retrieval device is particularly well suited for retrieving a percutaneous heart valve (e.g., for replacement of a native aortic, pulmonary, tricuspid or mitral valve) wherein a relatively smaller ID sheath is used to insert the balloon-mounted valve into the patient's vasculature. The retrieval device is compatible with delivery devices adapted for retrograde or antegrade delivery of such valves. In addition, the retrieval device may be used to facilitate retrieval of self-expanding prosthetic valves, such as valves including a shape memory stent or other support structure. Moreover, embodiments of the retrieval device can be used to retrieve other types of prosthetic valves or other implantable devices, such as balloon-expandable or self-expanding stents, that cannot be readily retracted back into an introducer sheath once inserted into a blood vessel.

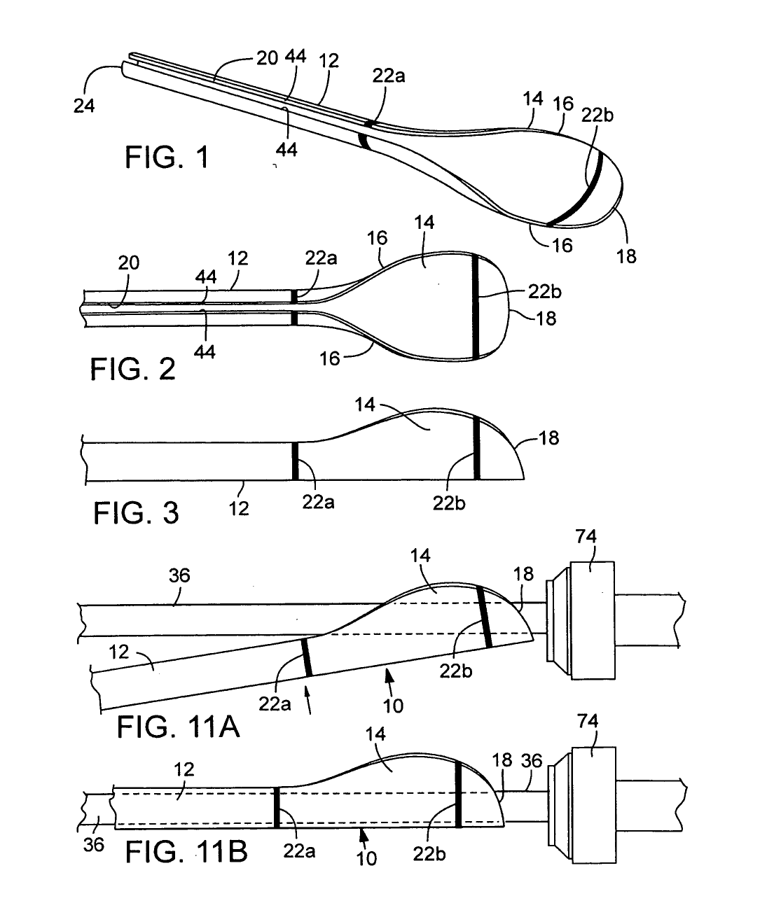

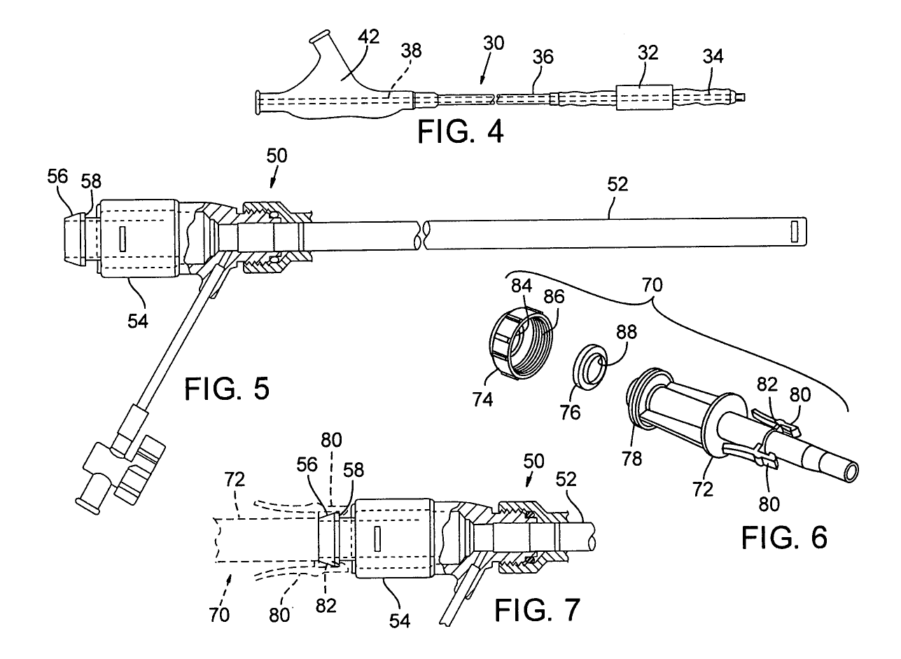

FIGS. 1-3 illustrate a retrieval device 10 that can be used to retract a balloon-mounted valve back into an introducer sheath, according to one preferred embodiment. The retrieval device 10 is adapted to be placed on and advanced over the shaft of a balloon catheter and through an introducer sheath assembly for retrieving a valve. FIG. 4, for example, schematically illustrates a typical conventional balloon catheter 30 that is used to deliver and deploy a percutaneous valve 32. FIGS. 5-7 illustrate an exemplary introducer sheath assembly 50 and loader assembly 70, which are used to insert the balloon catheter 30 into a patient's vasculature.

With reference again to FIG. 1, the illustrated retrieval device 10 includes an elongated shaft 12 and an enlarged distal end portion 14 extending from the distal end of the shaft 12. The distal end portion 14 is radially expandable and contractible between a contracted state in which the retrieval device can be inserted into and advanced through an introducer sheath and an expanded state as shown in FIG. 1-3 in which the distal end portion preferably has a diameter greater than the outer diameter of the non-deployed valve to be removed from the blood vessel. In the expanded state, the distal end portion can be placed in a position surrounding at least a portion of the non-deployed valve to facilitate retraction of the valve back into the introducer sheath, as described in detail below. The retrieval device 10 is preferably made of a flexible, resilient or self-expanding material so that the end portion 14 radially expands as it is advanced out of the introducer sheath. In certain embodiments, the retrieval device 10 is made of a polymeric material, such as high density polyethylene (HDPE), Teflon®, or any of various other suitable polymers.

The distal end portion 14 in the illustrated configuration is generally spoon shaped with side edges 16 that flare outwardly from the distal end of the shaft 12 and then curve inwardly so as to smoothly merge into a curved distal edge 18. The curved outer peripheral edges of the distal end portion 14 protect against vascular tissue damage as the retrieval device is advanced through the blood vessel. The shaft 12 is desirably formed with an elongated slot, or opening, 20 extending the entire length of the shaft to facilitate mounting of the retrieval device 10 on a catheter shaft, as described in detail below. The retrieval device also can include one or more radiopaque markers 22a, 22b to facilitate positioning of the retrieval device with respect to the valve to be removed from the blood vessel using conventional fluoroscopy.

The retrieval device 10 desirably is provided with an overall length from the proximal end 24 of the shaft 12 to the distal edge 18 that is greater than the combined lengths of the introducer sheath assembly 50 and the loader assembly 70 (FIGS. 5-7). This allows the proximal end portion of the shaft 12 to be used to advance the retrieval device 10 through the sheath assembly 50 until the distal end portion 14 extends past the distal end of the introducer sheath and into the blood vessel.

In one implementation, the retrieval device 10 is adapted for use in retrieving a 23-mm prosthetic aortic valve via a 22-French introducer sheath assembly. In this implementation, the retrieval device 10 is formed from a thin sheet of HDPE having a thickness of about 0.015 inch. In one preferred construction, the retrieval device has an overall length of about 22 inches, an outer diameter of about 0.230 inch, and an inner diameter of about 0.130 inch. The distal end portion has a maximum, expanded width (measured between side edges 16) of about 0.980 inch. Of course, these specific dimensions (as well as other dimensions provided in the present specification) are given to illustrate the invention and not to limit it. The dimensions provided herein can be modified as needed in different applications or situations.

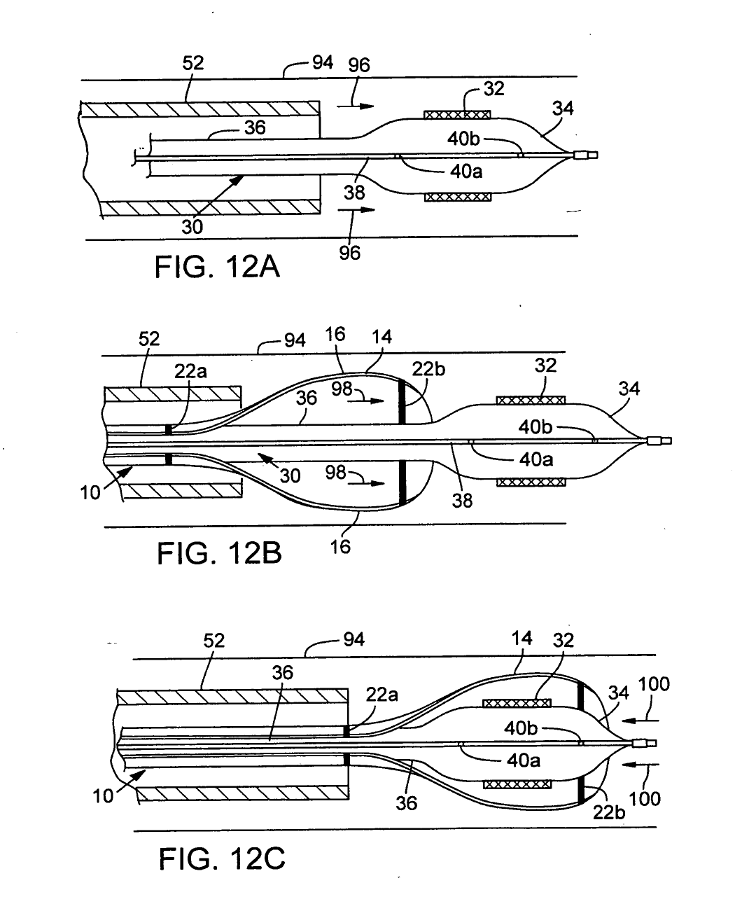

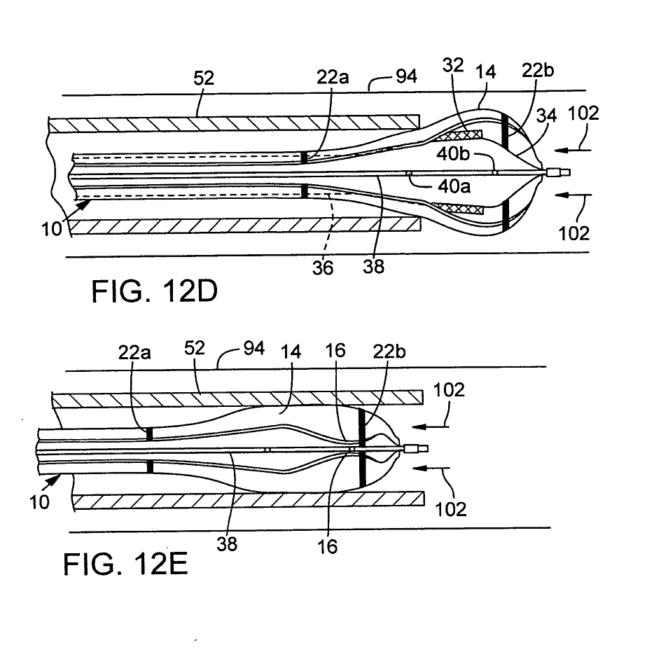

With reference to FIG. 4, the balloon catheter 30 includes an elongated, flexible main shaft 36, an inflatable balloon 34 coupled to the distal end of a main shaft 36, and a handle portion 42 coupled to the proximal end of the main shaft 36. The valve 32 is mounted in a crimped or collapsed state around the balloon 34. Extending co-axially through the main shaft 36 is a guidewire shaft 38 formed with a lumen for receiving a guidewire (not shown in FIG. 4). The guidewire is inserted first into the blood vessel and the balloon catheter 30 is advanced over the guidewire until the valve is positioned at the deployment site, as known in the art. To deploy the valve 32, a pressurized fluid from a pressurized fluid source (not shown) is introduced into the balloon 34, causing the balloon and the valve to expand. The balloon catheter 30 can include first and second radiopaque markers 40a, 40b (as shown in FIG. 12A) to assist in positioning the valve at the deployment site using conventional fluoroscopy.

The prosthetic valve 32 can take a variety of different forms. In particular embodiments, the valve generally comprises an expandable stent portion that supports a valve structure. The stent portion is constructed to have sufficient radial strength to hold the valve at the treatment site and resist recoil of the native valve leaflets. Additional details regarding exemplary balloon expandable valve embodiments can be found in U.S. Pat. Nos. 6,730,118 and 6,893,460, each entitled IMPLANTABLE PROSTHETIC VALVE, which are incorporated by reference herein.

The retrieval device 10 is adapted to be placed on and advanced over the catheter shaft 36 for retrieving the valve 32. As depicted in FIGS. 11A and 11B, the retrieval device 10 can be placed on the catheter shaft 36 by inserting the catheter shaft 36 through the longitudinal edges 44 of the slot 20 in the shaft 12. As the retrieval device 10 is pressed onto the catheter shaft 36, the shaft 12 exhibits sufficient flexibility and resiliency to expand around the catheter shaft 36 and substantially return to its normal shape surrounding the catheter shaft 36, thereby forming a “snap-fit” connection retaining the retrieval device 10 on the catheter shaft 36. The retrieval device 10 can be removed from the catheter shaft 36 by simply pulling the retrieval device away from the catheter shaft with sufficient force to allow the catheter shaft to slide through the opening 20 of the shaft 12. As used herein, a “snap-fit” arrangement or a “snap-fit” connection means a releasable connection between two bodies having opposing surfaces, which connection is formed by resiliently deforming at least one of the bodies so as to allow the opposing surfaces to be placed in an interlocking relationship with each other.

The shaft 12 of the retrieval device 10 desirably is sized to form a snug interference fit with the catheter shaft 36 with the entire inner surface of the shaft 12 contacting the outer surface of the catheter shaft 36 to prevent or at least minimize blood loss between the shaft 12 and the catheter shaft 36. The use of retrieval device 10 for removing the valve 32 from a patient is further described below.

The balloon catheter 30 is inserted into a blood vessel via an introducer sheath assembly, such as the exemplary introducer sheath assembly 50 shown in FIG. 6. The illustrated introducer sheath assembly 50 includes an introducer sheath 52 and an introducer housing 54 coupled to the proximal end of the introducer sheath 52. Introducer sheath diameters of 22 to 24 French typically are used in retrograde delivery of a prosthetic heart valve.

Attached to the proximal end of the introducer housing 54 is an end piece 56 having a central opening (not shown) in communication with the introducer housing 54 and a ridge 58 facing the distal end of the introducer housing 54. The introducer sheath 52 is adapted to be inserted into a blood vessel, with the introducer housing 54 located outside the blood vessel. The introducer sheath 52 desirably is coated with a hydrophilic coating. For retrograde delivery of a percutaneous heart valve, the sheath 52 preferably has a length of about 9 inches so as to extend just past the iliac bifurcation and into the abdominal aorta when inserted into a femoral artery.

A loader assembly, such as the loader assembly 70 shown in FIG. 6, can be used to insert and remove the balloon catheter 30 from the introducer sheath assembly 50 without substantial blood loss from the patient. As shown in FIG. 6, the illustrated loader assembly 70 includes a loader body 72, a removable loader cap 74, and a loader seal 76. The loader body 72 is generally tube shaped, having external threads 78 at a proximal end thereof for connection with the loader cap 74. The loader body 72 has a lumen extending the length thereof dimensioned to receive the catheter shaft 30, as further described below. The loader body 72 includes flexible flanges or arms 80 extending lengthwise of the body and having snap ridges 82 formed at the distal ends thereof. The loader cap 74 is formed with a central opening 84 in a proximal end thereof and a threaded inner surface 86 for engagement with the external threads 78 of the loader body 72. The loader seal 76 is sized to fit within the loader cap 74, and is formed with a central opening 88 that aligns with the loader cap opening 84.

The loader assembly 70 is adapted to be secured to and removed from the introducer housing 54 for inserting or removing the balloon catheter 30 from the introducer sheath assembly 50. In use, as shown in FIG. 7, a distal end portion 90 of the loader body 72 is inserted through the end piece 56 and into the introducer housing 54 until the flanges 80 of the loader body 72 snap onto the ridge 58 of the end piece, thereby securing the loader assembly to the introducer sheath assembly.

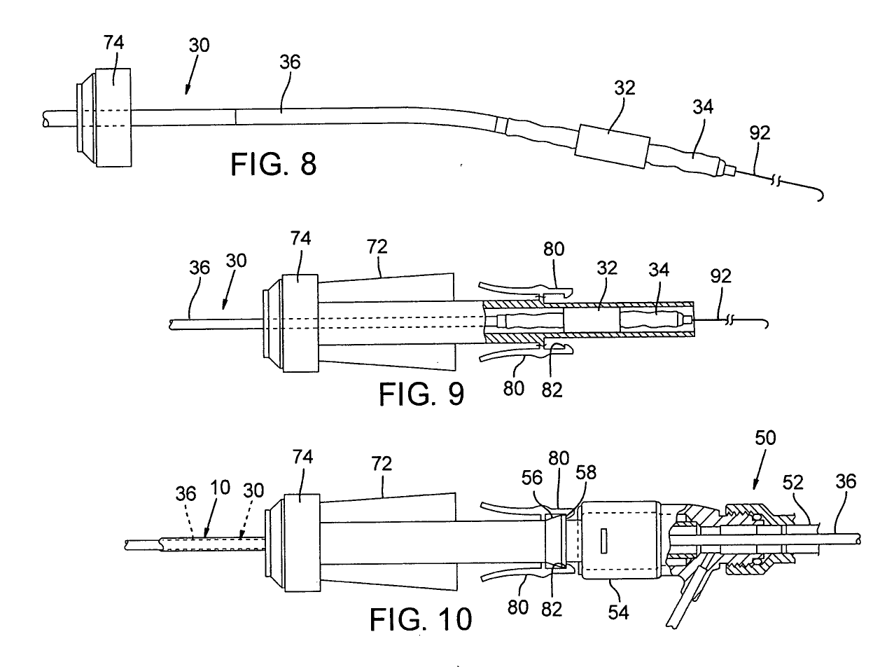

With reference to FIGS. 8-10, one preferred method of using the balloon catheter 30, the introducer sheath assembly 50, and the loader assembly 70 for percutaneous delivery of a prosthetic valve is illustrated. First, the blood vessel is dilated using a conventional dilator to allow the introducer sheath 52 to be inserted into the blood vessel followed by a guide wire 92. As shown in FIG. 8, the loader cap 74 and seal 76 are placed on the catheter shaft 36, which is placed over the guide wire 92. The distal end of the catheter shaft 36 mounting the crimped valve 32 is inserted into the proximal end of the loader body 72 and advanced through the lumen of the loader body and over the guide wire 92, as shown in FIG. 9. The loader cap 74 is then screwed onto the proximal end portion of the loader body 72.

With particular reference to FIG. 10, the catheter shaft 36 together with the loader assembly 70 are then inserted into the end piece 56 of the introducer sheath assembly 50 until the flanges 80 of the loader body 72 snap onto the ridge 58 of the end piece, securing the loader assembly to the introducer sheath assembly. As the loader body is inserted into the housing 54, the loader body passes through and causes internal valves (not shown) in the housing to open, thus placing the catheter shaft 36 in communication with the lumen of the introducer sheath 52 and the blood vessel.

As depicted in FIG. 12A, the valve 32, mounted on the distal end portion of balloon catheter 30, can then be advanced distally through the introducer sheath and into the blood vessel 94 (as indicated by arrows 96). In the example shown in FIG. 12A, when the valve 32 is advanced out of the sheath, the unconstrained valve expands slightly to an outer diameter that is about the same size as or slightly greater than the inner diameter of the introducer sheath 52, and therefore cannot be readily retracted back into the introducer sheath 52 if the valve cannot be successfully positioned at the deployment site. The retrieval device 10 facilitates retraction of the valve 32 back into the introducer sheath 52 such that the valve can be removed from the patient without invasive surgery. As described in more detail below, the retrieval device is configured to compress the prosthetic valve as the valve and retrieval device are withdrawn toward the sheath, thereby reducing the outer diameter of the valve. In addition or alternatively, the retrieval device is configured to align the prosthetic valve with the lumen of the sheath and direct the valve toward the distal opening as the valve is withdrawn into the sheath.

To mount the retrieval device 10 to the catheter shaft 36 for removing the valve 32, the retrieval tool 10 is press-fitted onto the catheter shaft 36 as previously described (FIGS. 11A and 11B). Because the retrieval device 10 is provided with longitudinal opening 20 extending the length of its shaft 12, the device can be easily placed on the catheter shaft 36 at any location along its length between the handle portion 42 of the balloon catheter 30 and the loader assembly 70.

The loader body 72 is then retracted from the introducer housing 54 and the loader cap 74 is unscrewed and removed from the loader body 72, after which the distal end portion 14 of the retrieval device 10 is advanced through the loader cap opening 84 and into the distal opening in the loader body 72. The loader cap 74 is then re-attached to the loader body 72 and the loader body is inserted into and re-connected to the introducer housing 54, as shown in FIG. 10. As depicted in FIG. 12B, the retrieval device 10 is then advanced distally through the introducer assembly 50 until the distal end portion 14 of the device is advanced out of the introducer sheath 52 and into the blood vessel 94 (as indicated by arrows 98). As noted above, the retrieval device desirably is of a length sufficient to allow the surgeon to grasp the proximal end portion of the retrieval device 10 and push it through the introducer assembly 50 until the distal end portion 14 extends out of the sheath 52.

Once advanced into the blood vessel, the distal end portion 14 assumes the expanded state shown in FIG. 12B. This allows the distal end portion 14 of the retrieval device to be placed in the position shown in FIG. 12C with the distal end portion 14 partially surrounding or extending over the valve 32, such as by retracting the balloon catheter proximally (as indicated by arrows 100). Conventional fluoroscopy can be used to determine the locations of markers 40a, 40b on the balloon catheter 30 relative to the markers 22a, 22b on the retrieval device 10 to position the valve 32 within the distal end portion 14 of the retrieval device. When the balloon catheter is retracted, the proximal edge of the valve 32 may snag or catch the distal edge 18 of the retrieval device 10. If this occurs, the retrieval device 10 can be rotated as necessary about its longitudinal axis to remove the distal edge 18 away from the proximal edge of the valve and permit the valve to be retracted within the distal end portion 14 of the retrieval device.

With reference to FIGS. 12D and 12E, after positioning the valve 32 within the distal end portion 14, the retrieval device 10 and the balloon catheter 30 are retracted together into the introducer sheath 52 in the proximal direction, as indicated by arrows 102. As shown, the distal end portion 14 of the retrieval device, rather than the valve 32, slides along the distal end and the inner surface of the introducer sheath 52 to prevent the valve 32 from snagging or catching the distal end of the introducer sheath as the valve is retracted into the introducer sheath. Accordingly, the retrieval device provides a collapsible transition member for reducing friction and preventing interference between the distal end of the introducer and the valve. Once inside the introducer sheath 52, the valve 32 and the balloon catheter 30 can be easily withdrawn from the blood vessel 92.

In the illustrated embodiment, the distal end portion 14 has an axial length that is greater than the length of the valve 32 and a maximum circumference (measured between side edges 16) that is slightly less than the outer circumference of the crimped valve 32 when the distal end portion is compressed around valve (as shown in FIG. 12E). This configuration allows the majority of the valve outer surface to be covered by the distal end portion 14 without the side edges 16 overlapping each other and increasing the outer diameter of the distal end portion 14. Maximizing the surface area of the distal end portion 14 contacting the valve 32 facilitates retraction of the valve into the introducer sheath and protects against trauma to the blood vessel 92.

In other embodiments, the retrieval device can be formed with a distal end portion that covers only a portion of the length and/or circumference of the valve.

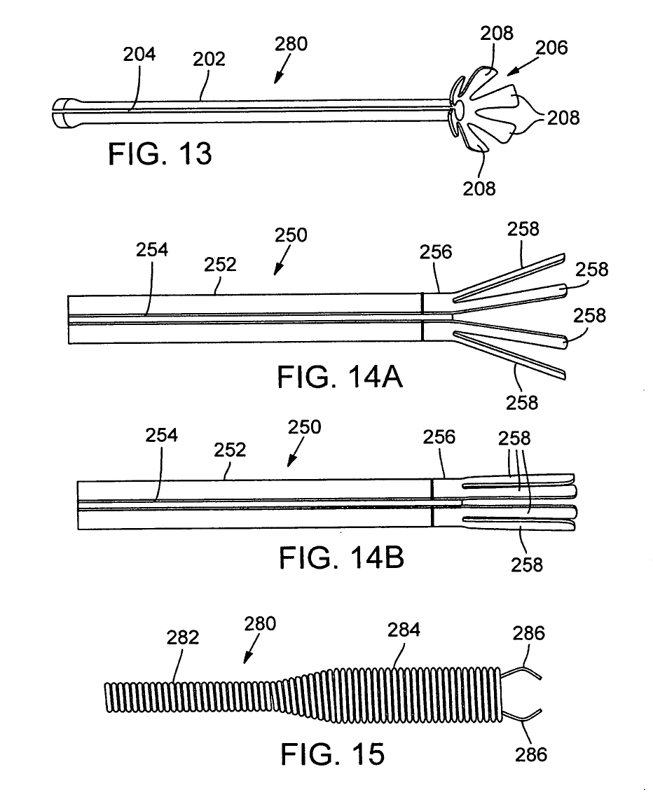

With reference to FIG. 13, a retrieval device 200 according to another preferred embodiment is illustrated. The retrieval device 200 includes an elongated shaft 202 that is split lengthwise to form a longitudinal opening 204 for placing the device on the catheter shaft 36. An enlarged distal end portion 206 of the retrieval device includes a plurality of angularly spaced fingers, or valve-engaging members, 208. The fingers 208 are preferably biased to assume a fanned out, expanded state as shown in FIG. 13, but exhibit sufficiently flexibility to flex or bend radially inwardly toward each other to a collapsed state for insertion into the introducer sheath 52. The retrieval tool 200 can have a one-piece, unitary construction (i.e., formed from a single piece of material) as shown and preferably is formed from a readily deformable material, such as any of various suitable polymeric materials, for snap-fitting the shaft 202 onto the catheter shaft 36.

The retrieval tool 200 functions in a manner similar to the retrieval tool 10 described above. In use, the retrieval tool 200 is inserted into the introducer assembly 50 and advanced distally until the distal end portion 206 extends out of the introducer sheath 52 and into the blood vessel, which causes the fingers 208 to move radially outwardly away from each other to the expanded state shown in FIG. 13. This allows a valve (e.g., valve 32 shown in FIGS. 12A-12E) to be positioned between the fingers 208. The fingers 208 can be sized to extend over the entire length of the valve or just a portion of the length of the valve when the valve is positioned between the fingers. As the tool 200 and the balloon catheter 30 are retracted together into the sheath 52, the fingers 52 collapse around the outer surface of the valve and allow the valve to be withdrawn back into the sheath 52.

With reference to FIGS. 14A and 14B, another embodiment of a retrieval device 250 is illustrated. The retrieval device 250 includes an elongated shaft 252 formed with a longitudinal opening 254 extending lengthwise of the shaft. The retrieval device 250 includes a distal end portion 256 made of a shape memory metal or metal alloy, such as NiTi (nickel titanium), coupled to the distal end of the shaft 252. The distal end portion 256 is formed with a plurality of longitudinally extending fingers, or valve-engaging members, 258 secured at their proximal ends to the device. The shaft 252 preferably is formed from a material that is readily deformable, such as any of various suitable polymeric materials, for snap-fitting the shaft 252 onto the catheter shaft 36.

The fingers 258 are preferably biased to assume the fanned out, expanded state shown in FIG. 14A, but exhibit sufficient flexibility to flex or bend radially inwardly toward each other to the collapsed state shown in FIG. 14B for insertion into the introducer sheath 52. The fingers 258 can be sized to extend over the entire length of the valve or just a portion of the length of the valve when the valve is positioned between the fingers. The retrieval device 250 can be used in substantially the same manner as described above for retrieval device 200 (FIG. 13).

With reference to FIG. 15, another alternative embodiment of a retrieval device 280 is illustrated. The retrieval device 280 in the form shown includes a flexible sheath 282 formed from a coiled wire and having an inner lumen sized to receive the catheter shaft 36 in a co-axial relationship. The sheath 282 alternatively can comprise a tubular member formed from a continuous piece of flexible material, rather than the illustrated coiled wire. The sheath 282 has an enlarged distal end portion 284 that houses a retractable grabbing device comprising two or more valve-engaging fingers 286. The valve-engaging fingers 286 are movable between a retracted position inside the distal end portion 284 of the sheath and an extended position outside of the distal end portion 284 as shown in FIG. 15. When extended out of the distal end portion 284, the valve-engaging fingers 286 expand radially outward from each other to allow a valve to be positioned between the valve-engaging fingers. The valve-engaging fingers 286 can be spring loaded such that user pressure on an operator switch is required to extend the valve-engaging fingers out of the distal end portion 284 and when user pressure is removed, a biasing force urges the valve-engaging fingers to the retracted position.

In use, the retrieval device 280 is placed on and advanced over the catheter shaft 36 through the introducer sheath 52. To retrieve a valve, the valve-engaging fingers 286 are extended out of the distal end portion 304 and placed over the valve. User pressure is removed from the operator switch to cause the valve-engaging fingers 286 to grasp or clamp onto the valve. The retrieval device 280, together with the valve, are then retracted back into the introducer sheath and removed from the body.

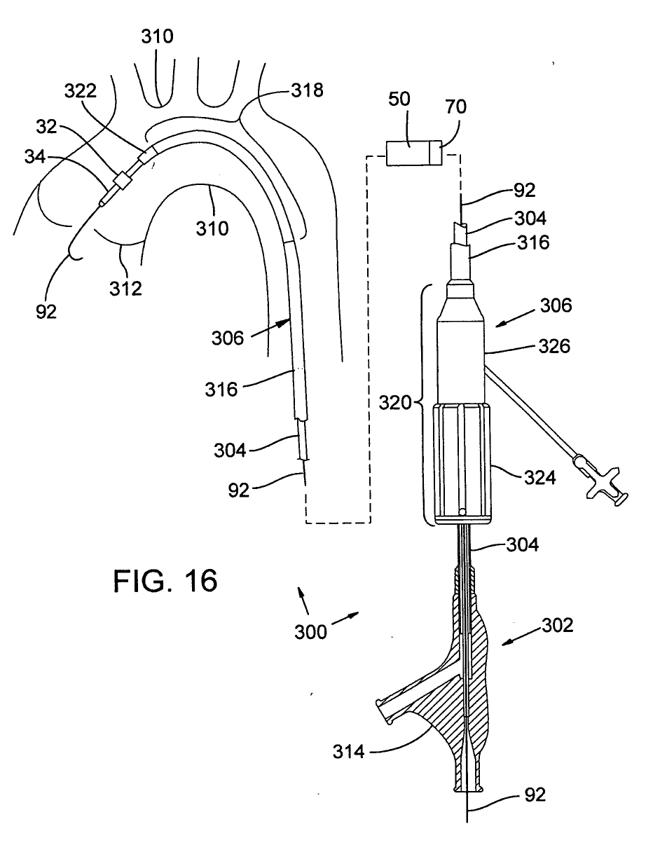

With reference to FIG. 16, a valve-delivery system 300 is illustrated that includes a balloon catheter 302 having a main catheter shaft 304 extending through an outer flexible catheter 306 (also referred to as a delivery sleeve assembly) that has a steerable portion 318 adjacent its distal end to help guide the balloon catheter through a blood vessel. The valve-delivery system 300 includes components of the balloon catheter 30, the introducer sheath assembly 50, and the loader assembly 70 shown in FIGS. 4-7. Thus, components in FIG. 16 that are identical to components in FIGS. 4-7 are given the same respective reference numerals. The valve-delivery system 300 is well suited for delivering a prosthetic valve 32 through a patient's vasculature over the aortic arch 310 to a location adjacent the diseased aortic valve 312, although the system also can be used to deliver prosthetic valves to other locations within the body.

The system 300 also can include a retrieval device, such as retrieval device 10 (FIGS. 1-3), retrieval device 200 (FIG. 13), retrieval device 250 (FIGS. 14A and 14B), or retrieval device 280 (FIG. 15). Unlike the previous embodiments, the retrieval device is adapted to be placed on the flexible catheter 306 for retracting a valve 32 back into the introducer sheath 52, rather than on the catheter shaft, if the valve 32 needs to be removed from the body. The system 300 can be used with the introducer sheath assembly 50 and the loader assembly 70 (shown schematically in FIG. 16) for inserting the balloon catheter 302 and the flexible catheter 306 into a blood vessel.

The balloon catheter 302 includes a balloon 34 coupled to the distal end of the catheter shaft 304 for mounting the prosthetic valve 32 and a handle or support portion 314 coupled to the proximal end of the catheter shaft 304. The flexible catheter 306 generally comprises an elongated, flexible sleeve, or shaft, 316 coupled at its proximal end to a handle, portion 320. The distal end of the sleeve 316 comprises the steerable portion 318 and a shroud 322 connected to the distal end of the steerable portion 318 adjacent the valve 32. The catheter shaft 304 extends generally co-axially through the handle portion 320, the sleeve 316, the steerable portion 318, and the shroud 322.

The handle portion 320 of the flexible catheter 306 includes an adjustable steering mechanism 324 and a hemostasis portion 326 coupled to the steering mechanism. The steering mechanism 324 is manually rotatable about its longitudinal axis to adjust the curvature of the steerable portion 318 via a pull wire (not shown) coupling the steering mechanism 324 to the steerable portion 318. In use, the balloon catheter 302 and the flexible catheter 306 are advanced together through the patient's vasculature to the deployment site of the valve 32 with the flexible catheter being used to adjust the curvature of the distal end portion of the catheter shaft 304 to assist in guiding or “steering” the valve 32 through the body. By adjusting the curvature of the steerable portion 318, retrograde advancement of the valve 32 can be achieved without damaging the aortic arch 310 or the valve 32. Depending on the experience of the operator, the valve can be advanced to the deployment site with little or no contact between the valve and the aorta. A more detailed description of the delivery system 300 is provided in co-pending U.S. application Ser. No. 11/238,853, which is incorporated herein by reference.

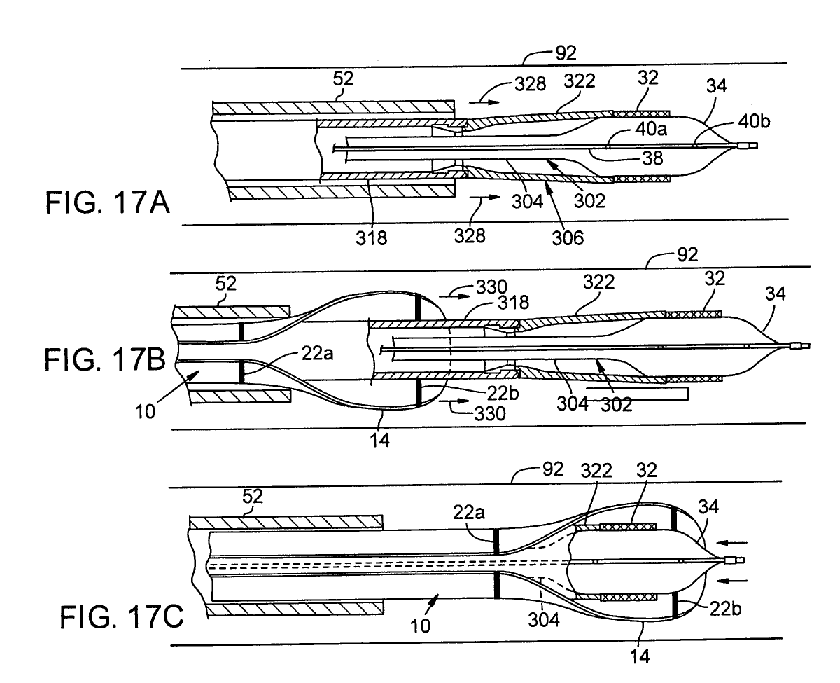

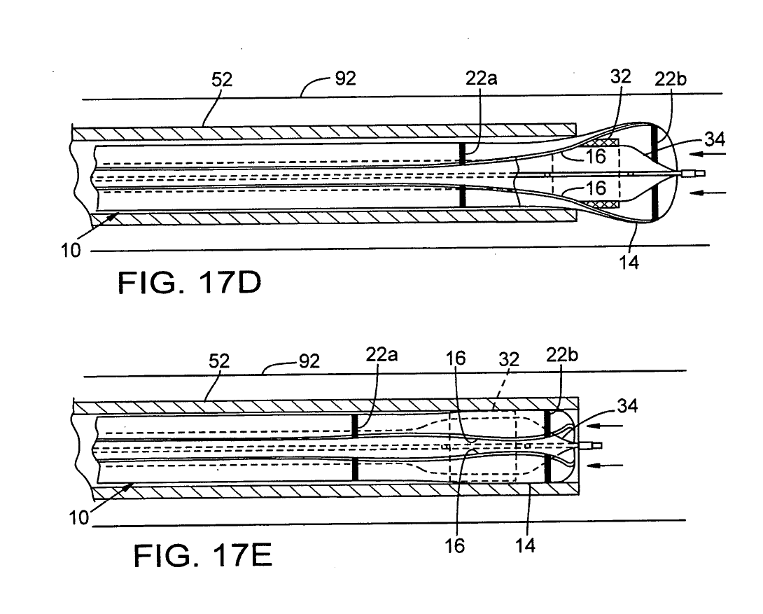

With reference to FIGS. 17A-17E, the operation of the retrieval device 10 with the delivery system 300 to retract the valve 32 back into the introducer sheath 52 will be described in more detail. As shown in FIG. 17A, the balloon catheter 302 and the flexible catheter 306 are advanced together through the introducer sheath 52 and into the blood vessel 92 for delivering the valve 32 to the deployment site. If the valve 32 needs to be removed after being inserted into the blood vessel, the loader assembly 70 is removed from the introducer assembly 50 and the loader cap 74 is removed from the loader body 72 (FIG. 6) as described above, and the retrieval tool 10 is placed on the sleeve 316 (FIG. 16) of the flexible catheter 306 at a location intermediate the handle portion 320 and the loader assembly 70 and advanced through the loader body 72 and into the introducer assembly 50. The loader cap 74 is re-attached to the loader body 72, which is inserted into and re-connected to the introducer assembly 50. The retrieval device 10 in this embodiment is configured to form a snug, interference fit with the outer surface of the sleeve 316 to minimize blood loss between the retrieval device and the sleeve 316.

The retrieval device 10 is advanced distally through the introducer sheath 52 and over the sleeve 316 and the steerable portion 318 until the distal end portion 14 of the retrieval device is advanced out of the introducer sheath in the direction of arrows 330, as depicted in FIG. 17B. The valve 32 is positioned within the distal end portion 14 of the retrieval device 10 (as depicted in FIG. 17C), after which the balloon catheter 302, the flexible catheter 306, and retrieval device 10 are retracted in the proximal direction to retract the valve 32 back into the introducer sheath 52 (as depicted in FIGS. 17D and 17E).

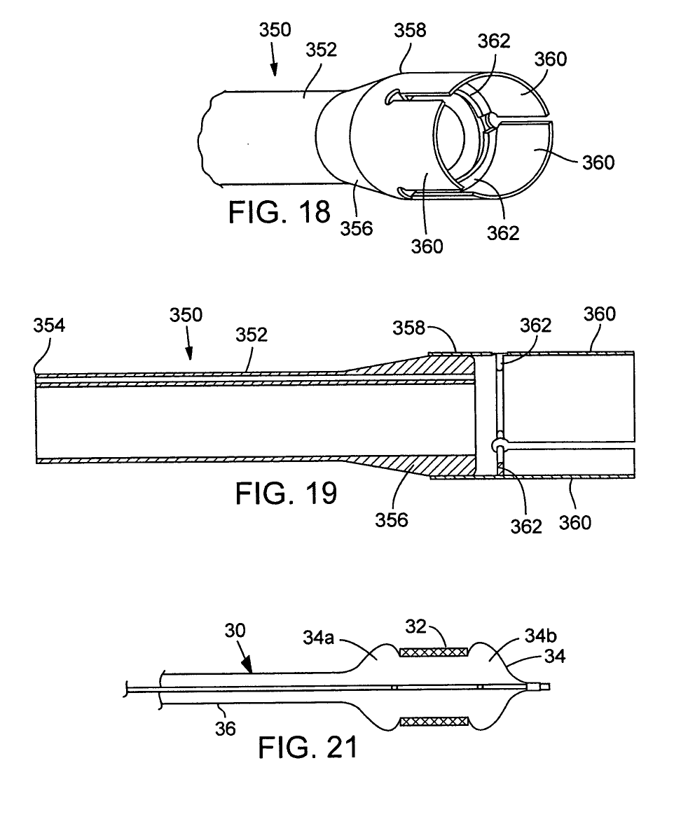

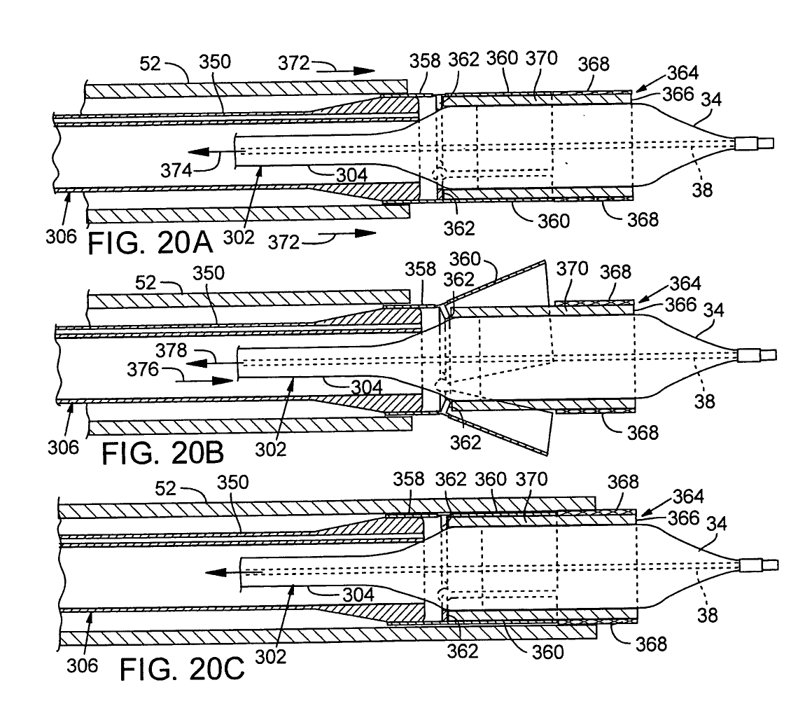

With reference to FIGS. 18-20, a retrieval device 350 according to another preferred embodiment can be coupled to the distal end of the steerable portion 318 of the flexible catheter 306 in place of the shroud 322 (FIGS. 20A-20C). This configuration provides a valve-retrieval mechanism integrated into the flexible catheter so that a separate retrieval device does not have to be mounted on the sleeve 316 and advanced through the introducer assembly 50 if and when it is desired to remove the valve 32.

In the embodiment shown in FIG. 20A-20C, a valve 364 is mounted to the balloon 34. The valve 364 has a valve body 366 and an outer skirt 368 surrounding the distal end portion of the valve body 366.

The retrieval device 350 comprises an elongated main body 352 having a proximal end portion 354 connected to the distal end of the steerable portion 318 and a distal end portion 356 connected to a valve-engaging mechanism 358. The valve-engaging mechanism 358 includes a plurality of longitudinally extending, arcuate fingers, or valve-engaging segments, 360 secured at their proximal end portions to the main body 352. The free ends (the distal ends) of the valve-engaging segments 360 are movable radially outwardly and inwardly between an expanded, generally funnel shaped arrangement (FIG. 20B) and a collapsed, generally cylindrical arrangement (FIGS. 18, 19 and 20A).

The valve-engaging segments 360 are preferably biased to assume the expanded, funnel shaped arrangement shown in FIG. 20B, and are movable radially inwardly toward each other to the collapsed state when an outside force is applied to the valve-engaging segments. Removal of the force allows the valve-engaging segments to revert back to the expanded state. The valve-engaging segments 360 can be formed from a resilient, shape-memory material (e.g., nickel titanium). Alternatively, separate biasing mechanism can be used for resiliently urging the valve-engaging segments to the expanded state. For example, each valve-engaging segment 360 can be connected to the main body 352 with a respective pre-tensioned hinge made of a resilient, deformable material configured to resiliently retain the valve-engaging segments in the expanded state.

Each valve-engaging segment 360 preferably includes a respective inner projection 362 adjacent the fixed end portion thereof extending radially inwardly from the inner surface of the valve-engaging segment and circumferentially along the inner surface. The projections 362 are preferably located to contact the proximal end of the valve 364 (FIG. 20A) when the valve is positioned between valve-engaging segments 360. Urging the valve 364 against the projections 362 causes the valve-engaging segments 360 to collapse around the valve.

The valve-engaging segments 360 in the illustrated configuration are sized to extend over a proximal portion 370 of the valve body 366 that is not surrounded by the skirt 368. When the valve 364 is introduced into a blood vessel via the introducer sheath 52, the balloon catheter 304 is advanced distally through the introducer sheath 52 with the valve-engaging segments 360 in the collapsed state surrounding portion 370 of the valve 364. To maintain the valve-engaging segments 360 in the collapsed state contacting the valve as the valve-engaging mechanism 358 and the valve 364 are advanced out of the introducer sheath (in the direction of arrows 372 in FIG. 20A) and through the blood vessel, slight pressure is applied to the balloon catheter 302 in the proximal direction (as indicated by arrow 374 in FIG. 20A) to maintain the valve 364 against the projections 362. Alternatively, the assembly can be provided with a mechanism for fixing the balloon catheter 302 against axial movement relative to the flexible catheter 306 to maintain the valve-engaging segments 360 in the collapsed state around the valve 364 as the catheters are advanced through the patient's vasculature to the deployment site.

At or adjacent the deployment site, the balloon catheter 302 is advanced distally relative to the flexible catheter 306 (as indicated by arrow 376 in FIG. 20B) to move the valve 364 outwardly from the valve-engaging mechanism 358 to allow the valve to be deployed in a conventional manner. If it becomes necessary or desirable to remove the non-deployed valve from the patient, the balloon catheter 302 is retracted in the proximal direction to retract the valve 364 into the valve-engaging mechanism 358 (as indicated by arrow 378 in FIG. 20B). As the proximal end of the valve is urged against the projections 362, the valve-engaging segments 360 are caused to collapse around portion 370 of the valve. The balloon catheter 302 and the flexible catheter 306 are then retracted together to retract the valve 364 back into the introducer sheath 52 (as shown in FIG. 20C).

In other embodiments, the valve-engaging segments 360 can be sized to extend over the entire length of the valve 364, rather than just a portion of the valve.

In an alternative approach for using the system shown in FIGS. 18-20, the retrieval device 350 can be maintained at a location adjacent the distal end of the introducer sheath 52 as the balloon catheter 302 is advanced through the patient's vasculature to the deployment site. When used in this manner, the flexible catheter need not be provided with steering mechanism for adjusting the curvature of the balloon catheter.

With reference to FIG. 21, yet another device and method for removing a non-deployed, percutaneous valve 32 from a patient's vasculature is illustrated. In this method, the balloon 34 may be partially inflated to distend the proximal and distal end portions 34a, 34b, respectively, of the balloon while causing little or no expansion of the valve 32. The end portions of the balloon are preferably distended to a diameter that is slightly greater than the outer diameter of the valve 32, as shown in FIG. 21. Thus, as the valve is retracted through the blood vessel, the partially inflated balloon 34 shields the ends of the valve to protect against trauma to the surrounding tissue. Accordingly, using the embodiment illustrated in FIG. 21, it may not be necessary to withdraw the valve back into the sheath before withdrawing the valve from the body. Rather, the balloon provides a protective member for preventing trauma to the inner wall of the blood vessel while withdrawing the valve through the vessel. In one variation of this method, the partially inflated proximal end portion 34a may be shaped to provide a transition member to facilitate withdrawing the valve back into the sheath. For example, the proximal end portion may be formed with a substantially conical shape. This embodiment is particularly well suited for systems wherein the valve diameter is smaller than the inner diameter of the sheath such that compression of the valve is not required. Rather, the proximal end portion of the balloon primarily provides a smooth transition member and centering mechanism that prevents the proximal end of the valve from interfering with the distal end of the introducer sheath, thereby facilitating retraction of the valve into the sheath.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A system for positioning and/or removing a prosthetic valve through a blood vessel, comprising:
an introducer sheath that is insertable into the blood vessel, the introducer sheath having a proximal end, a distal end, and a lumen;
a delivery catheter comprising an expandable distal end portion, the valve being mounted on the expandable distal end portion, the catheter being configured to be advanced through the introducer sheath and into the blood vessel to an implantation site; and
a valve removal device having a distal end portion configured to be advanced through the introducer sheath and into the blood vessel, the distal end portion being configured to be placed in a position at least partially surrounding the valve after the valve is advanced out of the introducer sheath and then retracted back into the introducer sheath along with the valve and the catheter.

2. The system of claim 1, wherein the distal end portion of the removal device is expandable such that when the distal end portion of the removal device is advanced out of the introducer sheath and into the blood vessel, the distal end portion expands to a diameter that is greater than an outer diameter of the valve.

3. The system of claim 1, wherein the removal device comprises an elongated, hollow shaft portion extending from the distal end portion of the removal device and adapted to be placed over a shaft of the catheter and advanced over the catheter shaft and through the introducer sheath.

4. The system of claim 3, wherein the shaft portion of the removal device is formed with an elongated opening extending the entire length of the shaft portion so that when the catheter shaft is inserted through the introducer sheath, the removal device can be placed on the catheter shaft by inserting the catheter shaft through the elongated opening in the shaft portion of the removal device.

5. The system of claim 4, wherein the shaft portion of the removal device conforms to the outer surface of the catheter shaft to minimize blood flow between the shaft portion and the catheter shaft.

6. The system of claim 4, wherein the shaft portion of the removal device is configured to be snap-fitted onto the catheter shaft.

7. The system of claim 2, wherein the distal end portion of the removal device is generally spoon shaped.

8. The system of claim 2, wherein the distal end portion comprises a plurality of longitudinally extending valve-engaging segments that are radially expandable and contractible about an outer surface of the valve.

9. The system of claim 2, wherein the distal end portion is made of a self-expanding material.

10. The system of claim 1, wherein the distal end portion of the removal device is configured to prevent the valve from contacting the introducer sheath when the distal end portion and the valve are retracted back into the introducer sheath.

11. The system of claim 1, wherein the valve defines an outer diameter that is equal to or greater than the inner diameter of the lumen of the introducer sheath when the valve is advanced out of the introducer sheath.

12. The system of claim 1, further comprising a steerable flexible catheter adapted to extend over the delivery catheter and being operable to adjust the curvature of at least portion of the delivery catheter, and wherein the removal device is connected to a distal end portion of the flexible catheter.

13. The system of claim 12, wherein the distal end portion of the removal device comprises a plurality of longitudinally extending valve-engaging members that are radially expandable and contractible toward and away from each other between an expanded position to allow at least a portion of the valve to be positioned within the valve-engaging members and a collapsed position with the valve-engaging members contacting said portion of the valve.

14. The system of claim 13, wherein the valve-engaging members are resiliently retained in the expanded position and are caused to move to the collapsed position when the valve is urged against inner surface portions of the valve-engaging members.

15. A valve-removal device for removing through a blood vessel a prosthetic valve mounted on a balloon of a balloon catheter after the valve and balloon are advanced into the blood vessel via an introducer sheath in the cardiovascular system, the device comprising an elongated shaft coupled to a valve-engaging distal end portion, the shaft being configured to be placed on a catheter shaft of the balloon catheter and advanced through the introducer sheath along the length of the catheter shaft, the distal end portion being configured to overlap at least a portion of the valve such that when the distal end portion and the valve are retracted together back into the introducer sheath assembly, the distal end portion shields the overlapped portion of the valve from the introducer sheath to facilitate retraction of the valve into the introducer sheath assembly.

16. The device of claim 15, wherein the valve-retrieval device has a length that is greater than the length of the introducer sheath to allow the device to be advanced or retracted through the introducer sheath by manually moving a proximal end portion of the shaft located outside of the introducer sheath.

17. The device of claim 15, wherein the shaft is split longitudinally the entire length of the shaft so that the device can be placed around the catheter shaft at any location along the length of the catheter shaft outside of the introducer sheath.

18. The device of claim 1, wherein the distal end portion expands to a diameter that is greater than an outer diameter of the valve when the distal end portion is advanced out of the introducer sheath.

19. A system for positioning and/or removing a prosthetic valve through a blood vessel, comprising:
a balloon catheter comprising a balloon at a distal end portion thereof, the valve being mounted on the balloon; and
introducing means for introducing the balloon catheter into the blood vessel; and
valve removal means for retracting the valve and the balloon back into the introducing means after the valve and the balloon are advanced through the introducing means into the blood vessel.

20. The system of claim 19, further comprising adjustment means for adjusting the curvature of a distal end portion of the balloon catheter, the valve removal means being connected to the adjustment means.

21. The system of claim 19, wherein the valve removal means comprises an elongated shaft adapted to be placed on a shaft of the balloon catheter in a co-axial relationship and advanced over the balloon catheter shaft through the introducing means, the valve removal means further comprising at least one valve-engaging member configured to engage an outer surface portion of the valve and prevent the outer surface portion of the valve from contacting the introducing means when the valve and the valve-engaging member are retracted into the introducing means.

22. A method for removing through a blood vessel a prosthetic valve mounted on a balloon of a balloon catheter after the valve and balloon are advanced into the blood vessel via an introducer sheath inserted partially into the blood vessel, the method comprising:
inserting the valve-removal device of claim 1 into the introducer sheath and advancing the valve-removal device until its distal end portion extends past the distal end of the introducer sheath and into the blood vessel;
covering at least a portion of the valve with the distal end portion of the valve-removal device; and
retracting the distal end portion and the valve together back into the introducer sheath.

23. A method of removing a prosthetic valve through a blood vessel, the valve being mounted on a balloon of a balloon catheter and having been inserted into the blood vessel via an introducer sheath inserted partially into the blood vessel, the method comprising:
advancing a removal device through the introducer sheath until at least a distal end portion of the removal device is advanced out of the introducer sheath;
covering at least a portion of the valve with the distal end portion of the removal device; and
retracting the distal end portion together with the valve back into the introducer sheath and removing the removal device and the balloon catheter from the blood vessel via the introducer sheath.

24. The method of claim 23, wherein the distal end portion of the removal device prevents the valve from contacting the introducer sheath when both are retracted into the introducer sheath.

25. The method of claim 23, further comprising expanding the distal end portion of the removal device when the distal end portion is positioned in the blood vessel and wherein the act of covering at least a portion of the valve comprises covering at least a portion of the valve with the expanded distal end portion.

26. The method of claim 25, wherein the distal end portion is self-expanding and therefore expands when advanced out of the introducer sheath.

27. The method of claim 25, wherein the distal end portion comprises a plurality of valve-engaging members that are radially expandable and contractible toward and away from each other between an expanded position and a collapsed position, wherein the act of expanding the distal end portion comprises expanding the valve-engaging members and the act of covering at least a portion of the valve comprises collapsing the valve-engaging members against said portion of the valve.

28. The method of claim 27, wherein the act of collapsing the valve-engaging members comprising urging the valve against inner surface portions of the valve-engaging members to cause the valve-engaging members to move to the collapsed position.

29. The method of claim 23, wherein the act of advancing a removal device through the introducer sheath comprises snap-fitting the removal device onto a shaft of the balloon catheter and sliding the removal device along the balloon catheter shaft through the introducer sheath.

30. A method of removing a prosthetic valve through a blood vessel, the valve being mounted on the balloon of a balloon catheter and having been inserted into the blood vessel via an introducer sheath inserted partially into the blood vessel, the method comprising:
partially inflating the balloon such that a proximal end portion and a distal end portion of the balloon not supporting the valve are distended to a diameter greater than the outer diameter of the valve; and
retracting the partially inflated balloon with the valve back into the introducer sheath.

31. The method of claim 30, wherein the act of partially inflating the balloon does not cause the valve to expand.
